(19) United States
(12) Patent Application Publication
(10) Pub. No.: US 2005/0203117 A1
(43) Pub. Date: Sep. 15, 2005

(54) Treating allergic and inflammatory conditions

(76) Inventor: Kim Allen Heithoff, Oldwick, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/124,445

(22) Filed: May 6, 2005

Related U.S. Application Data
(63) Continuation of application No. 10/088,629, filed on Mar. 19, 2002.

(51) Int. Cl. A61K 31/473

(52) U.S. Cl. 514/290

(57) ABSTRACT

Methods of using desloratadine for substantially returning work-related performance and workplace productivity of a person suffering from an allergic and/or inflammatory condition of the skin airway passages, e.g., season allergic rhinitis, pernninal allergic rhinitis, atopic dermatitis, urticaria or allergic asthma to the person's baseline work-related or baseline workplace productivity are disclosed.

### BACKGROUND OF THE INVENTION

This invention relates to substantially returning work-related performance and workplace productivity of a patient suffering from an allergic and/or inflammatory condition to the person's baseline work-related performance and workplace productivity by administering to such a patient an effective amount of desloratadine effective for such returning.

The symptoms and side effects of an allergic and/or inflammatory condition of the skin or upper and lower airway passages such as seasonal allergic rhinitis (“SAR”) include itchy, watery eyes, sneezing, runny nose, nasal congestion, urticaria, sommolence and general malaise. The pharmacologic effects of treating allergic and/or inflammatory condition such as SAR with sedating antihistamines include sommolence, blurred vision, dry mouth and individual performance impairment at home, in school and at work as well as impairment of workplace productivity. SAR affects up to 45 million people in the United States and many more millions worldwide.

Cockurn, Iain M, et al., in Business & Health, March 1999, pages 49-50 and in J Occup Eniviron Med., November 1999, Vol. 41(11), pages 948-953 disclose treating allergic reactions with sedating antihistamines, alone or in combination with decongestants, leads to impaired individual performance and decreased workplace productivity of workers compared to treatment with non-sedating antihistamines.

In view of the high prevalence of SAR, even relatively small effects on individual performance will have a significant impact on work-related performance and workplace productivity in the worldwide population, Thus, there is a need for a clinically more effective therapy for treating/preventing an allergic and or inflammatory condition of the skin and upper or lower airway passages in workers while simultaneously enhancing their work-related performance as well as their workplace productivity.

### SUMMARY OF THE INVENTION

The present invention provides a method of substantially returning the work-related performance of a person suffering from an allergic and/or inflammatory condition of the skin or airway passages to the person's baseline work-related performance which comprises administering an amount of desloratadine to said person effective for such returning.

The present invention provides a method of returning workplace productivity of a person suffering from an allergic and/or inflammatory condition of the skin or airway passages to the person's baseline workplace productivity which comprises administering an effective amount of desloratadine to said person effective for such returning.

In a preferred embodiment, the present invention provides a method of substantially returning work-related performance of a person suffering from seasonal allergic rhinitis to the person's baseline work-related performance which comprises administering an amount of desloratadine to such person effective for such returning.

In a preferred embodiment, the present invention provides a method of substantially returning workplace productivity of a person suffering from seasonal allergic rhinitis to the person's baseline workplace productivity which comprises administering an amount of desloratadine to said person effective for such returning.

In another preferred embodiment, the present invention provides a method of enhancing work-related performance of a patient suffering from atopic dermatitis or urticaria which comprises administering an amount of desloratadine effective for such enhancing.

In another preferred embodiment, the present invention provides a method of substantially returning workplace productivity of a person suffering from atopic dermatitis or urticaria to the person's baseline work-related performance to the person's baseline work-related performance which comprises administering an amount of desloratadine effective for such returning.

In another preferred embodiment, the present invention provides a method of returning performance of a person suffering from atopic dermatitis or urticaria to the person's baseline workplace productivity which comprises administering an amount of desloratadine to said person effective for such returning.

In another preferred embodiment, the present invention provides a method of substantially returning workplace productivity of a person suffering from an allergic and/or inflammatory condition of the skin or passages to the person's baseline workplace productivity by administering an initial amount of desloratadine to said person effective for such returning.

In another preferred embodiment, the present invention provides a method of substantially returning performance of a person suffering from an allergic and/or inflammatory condition of the skin or airway passages to the person's baseline workplace productivity by administering an initial amount of desloratadine to said person effective for such returning.

The invention also contemplates pharmaceutical compositions for substantially returning work-related performance and/or workplace productivity of a person suffering from an allergic and/or inflammatory condition of the skin or airway passage to the person's baseline work-related performance and/or workplace performance comprising an amount of desloratadine effective for such returning.

### DETAILED DESCRIPTION OF THE INVENTION

Persons afflicted with the symptoms and side effects of an allergic and/or inflammatory condition of the skin and upper or lower airway passages—such as seasonal allergic rhinitis—who are treated with an initial effective amount of desloratadine exhibit a significantly higher work-related performance and a significantly higher workplace productivity in a controlled clinical setting compared to untreated persons as well as with persons treated with an initial standard dose of the sedating antihistamine, diphenhydramine.

The phrase “the person's baseline work-related performance” as used herein means the person's work-related performance at a time prior to the person's exhibiting signs and/or symptoms of allergic and/or inflammatory conditions of the skin or airway passages as measured by art-recognized methods hereinafter described.

The phrase “the person's baseline workplace productivity” as used herein means the person's baseline workplace productivity as used herein means the person's performance at a time prior to the person's exhibiting the signs and/or symptoms of allergic and/or inflammatory conditions of the skin or airway passages as measured by art-recognized methods hereinafter described.

The phrase “substantially returning” as used herein in reference to a person's baseline work-related performance or baseline workplace productivity means returning to within about 5-10%, preferably within about 5% and more preferably within about 1-2% of the baseline values.

The phrase “allergic and/or inflammatory conditions of the skin or airway passages” as used herein means those allergic and/or inflammatory conditions and symptoms found on the skin and in the airway passages from the nose to the lungs. Typical allergic and/or inflammatory conditions of the skin and upper and lower airway passages include seasonal and perennial allergic rhinitis, non-allergic rhinitis, asthma including allergic and non-allergic asthma, sinusitis, colds (in combination with a NSAID, e.g., aspirin ibuprofen or APAP) and/or a decongestant e.g. pseudoephedrine), dermatitis, especially allergic and atopic dermatitis, and urticaria and symptomatic dermographism as well as retinophathy, and small vessel diseases, associated with diabetes mellitus.

The amount of desloratadine effective for treating or preventing allergic and/or inflammatory conditions of the skin and upper and lower airway passages will vary with the age, sex, body weight and severity of the allergic and inflammatory condition of the patient. Typically, the amount of desloratadine effective for treating or preventing such allergic and inflammatory conditions is in the range of about 2.5 mg/day to about 45 mg/day, preferably about 2.5 mg/day to about 20 mg/day, or about 4.0 mg/day to about 15 mg/day, or about 5.0 mg/day to about 10 mg/day, more preferably about 5.0 mg/day to about 7.5 mg/day, and most preferably about 5.0 mg/day in single or divided doses, e.g. two 2.5 mg doses, or about 5.0 mg/day in a single dose.

Desloratadine is a non-sedating long acting histamine antagonist with potent selective peripheral H1-receptor antagonist activity. Following oral administration, loratadine is rapidly metabolized to descarboethoxyloratadine or desloratadine, a pharmacologically active metabolite. In vitro and in vivo animal pharmacology studies have been conducted to assess various pharmacodynamic effects of desloratadine and loratadine. In assessing antihistamine activity in mice (comparison of ED₅₀ value), desloratadine was relatively free of producing alterations in behavior alterations in behavior, neurologic or autonomic function. The potential for desloratadine or loratadine to occupy brain H1-receptors was assessed in guinea pigs following i.p. administration and results suggest poor access to central histamine receptors for desloratadine or loratadine.

In vivo studies also suggest that an inhibitory effect of desloratadine on allergic bronchospasm and cough can also be expected.

The clinical efficacy and safety of desloratadine has been documented in over 3,200 seasonal allergic rhinitis patients in 4 double-blind, randomized clinical trials. The results of these chemical studies demonstrated the efficacy of desloratadine in the treatment of adult and adolescent patients with seasonal rhinitis.

Desloratadine is particularly useful for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal allergic rhinitis, including nasal congestion, in patients in need of such treating and/or preventing. Desloratadine may be used alone, or in combination with a decongestant, e.g., pseudeoephridine and/or an analgesic, e.g., a NSAID such as acetominophen or ibuprofen.

### Study Designs and Concepts

A series of randomized, double-blinded (treatment), placebo-controlled studies have been designed to quantify the impact of seasonal allergic rhinitis (“SAR”) and SAR treatments on work-related performance and workplace productivity of subjects as measured by art-recognized selected areas of performance and workplace productivity. In one series of studies, the effects of SAR (burden of disease) in subjects will be quantified by comparing the work-related performance levels in asymptomatic SAR subjects to the work-related subjects performance levels in symptomatic SAR subjects. In another series of studies, the differential impact following two different treatments for SAR on work-related performance of subjects will be quantified: the effects of desloratadine 5 mg tablets will be compared to diphenhydramine 50 mg (and placebo of each drug) among subjects with symptomatic SAR during exposure of the subjects to ragweed pollen. A consistent level of ragweed pollen exposure will be assured by conducting these studies in an environmental exposure unit (EEU). The baseline work-related performance and baseline workplace productivity of each subject will be measured at day 0 prior to exposure to ragweed pollen in the EEU.

### Work-Related Performance Tests

The work-related performance abilities of the subjects to be examined in one study series were selected based on the consensus of an expert panel consisting of neuropsychologists, industrial psychologists, and allergists. These work-related performance abilities cover the domains thought to be most affected by the symptoms of SAR and/or by sedation caused by SAR treatments. In addition, the expert panel prioritized those performance domains that are most closely related to abilities associated with safety and productivity. The work-related performance abilities were then mapped by the expert panel to neuropyschological performance tests.

Primary Endpoint:

The effects of SAR (also called the burden of disease) will be measured by measuring the selective attention in asymptomatic versus symtomatic subjects and in symptomatic subjects treated with desloratadine 5 mg tablets versus symptomatic subjects treated diphenhydramine 50 mg.

| Performance Domain | Definition | Performance Measure |
|---|---|---|
| Selective Attention | The ability to | Kay Continuous |
| | concentrate and not be | Performance Test |
| | distracted while | (Omission Errors Score) |
| | performing a task over | |
| | a period of time. | |

Secondary Endpoints:

1. Impact of Treatment (Desloratadine vs. Diphenhydramine) will be determined by measuring the perceptual speed in asymptomatic versus symtomatic subjects and in symptomatic subjects treated with desloratadine 5 mg tablets versus symptomatic subjects treated diphenhydramine 50 mg.

| Performance Domain | Definition | Performance Measure |
|---|---|---|
| Perceptual Speed | The ability to quickly | Automated |
| | and accurately compare | Neuropsychological |
| | letters, numbers, | Matrices (ANAM) |
| | objects, pictures or | Running Memory CPT |
| | patterns. The things to | (Accuracy Score) |
| | be compared may be | |
| | presented at the same | |
| | time or one after the | |
| | other. This ability also | |
| | includes comparing a | |
| | presented object with a | |
| | remembered object | |
| Near Vision | The ability to see details | CogScreen Visual |
| | of objects at a close | Sequence Comparison |
| | range (within a few feet | (Accuracy Score) |
| | of the observer). | |

2. Burden of Disease will be measured in asymptomatic vs. symptomatic subjects; and in symptomatic subjects vs. those treated with Desloratadine by measuring the information ordering as follows:

| Performance Domain | Definition | Performance Measure |
|---|---|---|
| Information Ordering | The ability to follow a | CogScreen Digit |
| | given set of rules or | Symbol Coding (with |
| | instructions in order to | Delay) (Response Time |
| | arrange things or | Score) |
| | actions in a certain | |
| | order. The things or | |
| | actions can include | |
| | numbers, letters, | |
| | words, pictures, | |
| | procedures, sentences, | |
| | and mathematical or | |
| | logical operations. | |

3. Other Endpoints:

Additional measures of some of the performance domains will also be included as secondary endpoints. These include, but are not limited to, problem sensitivity, memorization, number facility, time sharing, and response orientation, and rate control.

### Inclusion and Exclusion Criteria

Finally, standard inclusion and exclusion criteria will be used to assure that other factors, such as nicotine and/or alcohol use or sleep disturbances, are not contributing to any observed effect.

### Environmental Exposure Unit (EEU)

The EEU is a scientifically recognized pollen exposure system that has been used to evaluate the efficacy of anti-allergic medications, including determinations of the “onset of action” of these medications to relieve the signs and symptoms of pollen-induced allergic rhinitis. The controlled exposure to an aeroallergen, usually short ragweed pollen, has eliminated variables associated with other methods of clinical evaluation of these medications. The clinical relevance of the results of this test system have been validated by comparison of the results of clinical trails in this unit with those of other modes of allergen challenge, in particular exposure of allergic subjects to natural environmental increases in pollen levels.

Prior to those study days when the subjects are to be symptomatic and will undergo work-related performance and work-place productivity testing, they will be exposed during two to six priming sessions of 3 hours each to controlled pollen levels (3500±500 grains/m³) in the EEU. Subjects will record symptom severity every 30 minutes until the symptom severity criteria for enrollment in the study are met or the 3 hours have lapsed following which they will be transferred to a pollen-free room for up to one hour of observation. Subjects whose symptoms are so severe that they cannot remain in the EEU for at least 3 hours are moved to a pollen-free room and discharged from the study. To qualify for enrollment the subjects are required to achieve a total SAR symptom severity score of ≧10 made up of a nasal symptom score of ≧6 and of ≧4 for the non-nasal symptoms. On leaving the EEU those subjects who meet the severity scores inclusion criteria will be assigned to computer-generated randomization.

On the Baseline (symptomatic) and treatment-study days the enrolled subjects will report to the EEU at 7:30 AM. They will complete the daily baseline pre-exposure evaluatin of their SAR symptom severity at 8:00 AM, following which they will begin exposure to ragweed pollen (3500±500 grain/m³) for 8 hours, i.e., from 8:00 AM to 4:00 PM. Promptly following symptom severity ratings at 9:30 AM, the subjects will be evaluated for qualification for dosing and continuation in the study. Immediately after completing the 10:00 AM dairy card, all subjects will take their medications with a glass (180 mL) of water.

The work-related performance and work-place productivity testing will begin approximately 1½ hours after the initial dosing and will continue until approximately 2 hours after the initial dosing. This timing will allow for testing to be completed during the time that the two drugs are expected to show efficacy.

### Work-Place Productivity Tests

The work-place productivity tests selected will be based on their sensitivity to the effects of sedation and seasonal allergic rhinitis symptoms, and their relevance to the skills required for word processing. The same subject inclusion/exclusion criteria used for the work-related performance studies will be used. A consistent level of ragweed pollen exposure will be assured by conducting these studies in the above-described environmental exposure unit (EEU).

Primary Study Objective:

To show that work-place productivity is higher when subjects with symptomatic SAR are treated with desloratadine, 5 mg tablets antihistamine, than when subjects are treated with diphenhydramine 50 mg, a sedating antihistamine after exposure of both sets of subjects to ragweed pollen in an above-described EEU.

Secondary Study Objectives:

1. To show that work-related performance and workplace productivity are higher when subjects with symptomatic SAR are treated with desloratadine than when they are not treated; and
2. To show that SAR negatively impacts workplace productivity.

### Research Background for the Studies

The hypotheses that relate to the objectives for these studies are based on the documented findings that dosing with diphenhydramine causes somnolence and impairment of cognitive and psychomotor functions and vigilance and intuitive projections, and that the signs and symptoms of SAR adversely affect those same functions. SAR may exert its impairing effects not only by affecting visual and auditory responses and upper airway breathing capacity but also by a sense of general malaise and discomfort. These impairments of work-related performance should result in diminished workplace productivity.

The study subjects, who will have a history of ragweed pollen associated SAR and a documented positive skin test to short ragweed pollen, will be evaluated while asymptomatic and symptomatic to establish baseline work-related performance and workplace productivity data to meet the study objectives. Because these subjects will be evaluated for the effects of their SAR signs and symptoms and of the two study medications on individual performance and workplace productivity, they will need to meet at least minimal requirements for typing/word processing skills.

Both medications (desloratadine and diphenhydramine) are expected to relieve the signs and symptoms of SAR during the course of the treatment study day, beginning as soon as one-and-one half-hours after dosing and continuing during the testing periods.

### General Experimental

U.S. Pat. No. 4,659,716 discloses desloratadine as a non-sedating antihistamine as well as methods of making desloratadine, pharmaceutical compositions containing it and methods of using desloratadine and pharmaceutical compositions containing it to treat allergic reaction in mammals.

U.S. Pat. No. 5,595,997 discloses pharmaceutical compositions containing desloratadine and methods of using desloratadine for treating allergic rhinitis.

Desloratadine is available from Schering Corporation, Kenilworth, N.J. Diphenhydramine is available under the BENADRYL trademark on a non-prescription basis.

The pharmaceutical compositions of desloratadine be adapted for any mode of administration e.g., for oral, parenteral, e.g., subcutaneous (“SC”), intramuscular (“IM”), intravenous (“IV”) and intraperitoneal (“IP”), topical or vaginal administration or by inhalation (orally or intranasally). Preferably desloratadine is administered orally.

Such compositions may be formulated by combining desloratadine or an equivalent amount of a pharmaceutically acceptable salt thereof with a suitable, inert, pharmaceutically acceptable carrier or diluent which may be either solid or liquid. Desloratadine may be converted into the pharmaceutically acceptable acid addition salts by admixing it with an equivalent amount of a pharmaceutically acceptable acid. Typically suitable pharmaceutically acceptable acids include the mineral acids, e.g., HNO₃, H₂SO₄, H₃PO₄, HCl, HBr, organic acids, including, but not limited to, acetic, trifluoroacetic, propionic, lactic, maleic, succinic, tartaric, glucuronic and citric acids as well as alkyl or arylsulfonic acids, such as p-toluenesulfonic acid, 2-naphthalenesulfonic acid, or methanesulfonic acid. The preferred pharmaceutically acceptable salts are trifluoroacetate, tosylate, mesylate, and chloride. Desloratadine is more stable as the free base than as an acid addition salt and the use of the desloratadine free base in pharmaceutical compositions of the present invention is more preferred.

Solid form compositions include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Desloratadine may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical composition is in a unit dosage form. In such form, the preparation is subdivided into suitably sized unit doses containing appropriate quantities of desloratadine and other, if any active component, e.g., effective amounts to achieve the desired purpose.

1. A method of substantially returning work-related performance and/or workplace productivity of a person suffering from an allergic and/or inflammatory condition of the skin or airway passages to the person's baseline work-related performance and/or workplace productivity by administering to said person an amount of desloratadine effective for such enhancing.

2. The method of claim 1 wherein the amount of desloratadine is about 2.5 mg/day to about 45 mg/day.

3. The method of claim 1 wherein the amount of desloratadine is about 5 mg/day to about 15 mg/day.

4. The method of claim 1 wherein the amount of desloratadine is about 5 mg/day to about 10 mg/day.

5. The method of claim 1 wherein the amount of desloratadine is about 5 mg/day.

6. The method of claim 1 wherein the allergic and/or inflammatory condition of the skin or airway passages is season allergic rhinitis, pernninal allergic rhinitis, atopic dermatitis, urticaria or allergic asthma.

7. A method of substantially returning work-related performance of a person suffering from an allergic and/or inflammatory condition of the skin or airway passages to the person's baseline work-related performance by administering to said person an amount of desloratadine effective for such returning.

8. The method of claim 7 wherein the amount of desloratadine is about 2.5 mg/day to about 45 mg/day.

9. The method of claim 7 wherein the amount of desloratadine is about 5 mg/day to about 15 mg/day.

10. The method of claim 7 wherein the amount of desloratadine is about 5 mg/day to about 10 mg/day.

11. The method of claim 7 wherein the amount of desloratadine is about 5 mg/day.

12. The method of claim 7 wherein the allergic and/or inflammatory condition of the skin or airway passages is season allergic rhinitis, pernninal allergic rhinitis, atopic dermatitis, urticaria or allergic asthma.

13. A method of substantially returning workplace productivity of a person suffering from an allergic and/or inflammatory condition of the skin or airway passages to the person's baseline workplace productivity by administering to said person an amount of desloratadine effective for such returning.

14. The method of claim 13 wherein the amount of desloratadine is about 2.5 mg/day to about 45 mg/day.

15. The method of claim 13 wherein the amount of desloratadine is about 5 mg/day to about 15 mg/day.

16. The method of claim 13 wherein the amount of desloratadine is about 5 mg/day to about 10 mg/day.

17. The method of claim 13 wherein the amount of desloratadine is about 5 mg/day.

18. The method of claim 13 wherein the allergic and/or inflammatory condition of the skin or airway passages is season allergic rhinitis, pernninal allergic rhinitis, atopic dermatitis, urticaria or allergic asthma.

19. A method of substantially returning work-related performance of a person suffering from seasonal or perennial allergic rhinitis to the person's baseline work-related performance which comprises administering an amount of desloratadine to said person effective for such returning.

20. The method of claim 19 wherein the amount of desloratadine is in the range of about 2.5 mg/day to about 45 mg/day.

21. The method of claim 19 wherein the amount of desloratadine is about 5 mg/day to about 15 mg/day.

22. The method of claim 19 wherein the amount of desloratadine is about 5 mg/day to about 10 mg/day.

23. The method of claim 19 wherein the amount of desloratadine is about 5 mg/day.

24. A method of substantially returning workplace productivity of a person suffering from seasonal or perennial allergic rhinitis which comprises administering an amount of desloratadine to said person effective for such returning.

25. The method of claim 24 wherein the amount of desloratadine is in the range of about 2.5 mg/day to about 45 mg/day.

26. The method of claim 24 wherein the amount of desloratadine is about 5 mg/day to about 15 mg/day.

27. The method of claim 24 wherein the amount of desloratadine is about 5 mg/day to about 10 mg/day.

28. The method of claim 24 wherein the amount of desloratadine is about 5 mg/day.

29. A method of substantially returning work-related performance of a person suffering from atopic dermatitis or urticaria to the person's baseline work-related performance which comprises administering an amount of desloratadine to said person effective for such returning.

30. The method of claim 29 wherein the amount of desloratadine is about 2.5 mg/day to about 45 mg/day.

31. The method of claim 29 wherein the amount of desloratadine is about 5 mg/day to about 15 mg/day.

32. The method of claim 29 wherein the amount of desloratadine is about 5 mg/day to about 10 mg/day.

33. The method of claim 29 wherein the amount of desloratadine is about 5 mg/day.

34. The method of claim 29 wherein the patient is suffering from atopic dermatitis.

35. The method of claim 29 wherein the patient is suffering from urticaria.

36. A method of substantially returning workplace productivity of a person suffering from atopic dermatitis or urticaria to the person's baseline workplace productivity which comprises administering an amount of desloratadine to said person effective for such returning.

37. The method of claim 36 wherein the amount of desloratadine is about 2.5 mg/day to about 45 mg/day.

38. The method of claim 36 wherein the amount of desloratadine is about 5 mg/day to about 15 mg/day.

39. The method of claim 36 wherein the amount of desloratadine is about 5 mg/day to about 10 mg/day.

40. The method of claim 36 wherein the amount of desloratadine is about 5 mg/day.

41. The method of claim 36 wherein the patient is suffering from atopic dermatitis.

42. The method of claim 36 wherein the patient is suffering from urticaria.

43. A method of substantially returning work-related performance of a person suffering from an allergic and/or inflammatory condition of the skin or lower airway passages to the person's baseline work-related performance by administering to said person an initial amount of desloratadine effective for such returning.

44. The method of claim 43 wherein the amount of desloratadine is about 5 mg/day.

45. A method of substantially returning workplace productivity of a person suffering from an allergic and/or inflammatory condition of the skin or lower passages to the person's baseline workplace productivity by administering an initial amount of desloratadine to said person effective for such returning.

46. The method of claim 45 wherein the amount of desloratadine is about 5 mg/day.
